(19) United States
(12) Patent Application Publication
(10) Pub. No.: US 2018/0369020 A1
(43) Pub. Date: Dec. 27, 2018

(54) PROVIDING OPHTHALMIC LASER PULSES IN A FAST ARRAY

(71) Applicant: LUMENIS LTD., Yokneam (IL)

(72) Inventors: Alon Shacham, Katzir (IL); Anthony Jason Mirabito, Derry, NH (US)

(21) Appl. No.: 16/016,989

(22) Filed: Jun. 25, 2018

Related U.S. Application Data
(60) Provisional application No. 62/524,708, filed on Jun. 26, 2017.

(51) Int. Cl. A61F 9/008; H01S 3/101

(52) U.S. Cl. CPC A61F 9/00823; A61F 2009/00863; H01S 3/101

(57) ABSTRACT

A method of treating ophthalmic tissues of the body by the application of multiple laser pulses on each single spot intended to be treated while reducing heating of the ophthalmic tissues includes providing a source of pulsed laser energy movable in X and Y dimensions; determining the location and dimensions of the area of the ophthalmic tissue to be treated; determining an array of X rows by Y columns target spot positions within the area to be treated; then firing a single pulse of the source of laser energy at an initial target spot in the first of the X rows; firing a single second spot of laser energy at the next adjacent target spot along the first of the X rows and repeating the sequence until treatment is completed. A continuous wave (CW) laser energy source may also be utilized in practicing the method by moving the CW laser energy source in a determined pattern.

### RELATED APPLICATIONS

This application claims priority to U.S. Provisional Application Ser. No. 62/524,708, filed Jun. 26, 2017, the entirety of which disclosure is herein incorporated by reference.

### FIELD OF THE INVENTION

The present application relates to ophthalmic treatment of the eyes using a laser source.

### BACKGROUND OF THE INVENTION

In ophthalmic applications of laser radiation to eye tissue it is important that excessive heating up of the eye tissue be avoided to prevent more damage being done to the tissue than needed for therapeutic effects and do the treatment fast while the patient eye does not move. The present invention relates to addressing a methodology to eliminate or reduce tissue damage using already existing equipment and technologies.

While it is known to move a laser energy application device in aesthetic applications for skin tissue in a fashion (as seen in US 2014/0121730) to eliminate sequential applications of laser energy to adjacent spots, the present invention is directed to treating eye tissues which are the much more delicate and subject to more damage than the more robust skin tissue such as found in the arms or the legs or even the face.

### SUMMARY

In an aspect, a method of treating ophthalmic tissues of the retina by the application of multiple subthreshold laser pulses on each single spot intended to be treated while reducing heating of the ophthalmic tissues, includes: providing a source of pulsed laser energy with energy output in the microsecond regime, wherein the pulsed output laser pulse movable in two dimensions; determining the location and dimensions of an area of the retina tissue to be treated; determining an array of n target spot positions within the area of the retina to be treated; targeting a first spot position with one pulse, then targeting the next n spot positions with one pulse until all n spot positions have received one pulse and then restarting the sequence at the first spot position and, repeating the sequence above X number of times until treatment is completed. A galvo-mirror apparatus may be utilized to move the laser in the two directions.

In another aspect, the method further includes a programmable controller: the controller is configured to control the on and off times of the laser. The method further comprises the controller moving the laser from a spot position to a subsequent spot position during the off time of the laser and activating the laser after movement to the subsequent spot; thereby, the treatment time is reduced from beginning to completion.

In a further aspect, a method of treating ophthalmic tissues of the retina by the application of multiple subthreshold laser pulses on each single spot intended to be treated while reducing heating of the ophthalmic tissues, includes: providing a source of pulsed laser energy with energy output in the microsecond regime, wherein the pulsed output laser pulse movable in two dimensions; determining the location and dimensions of an area of the retina tissue to be treated; determining an array of X by Y target spot positions within the area of the retina to be treated; firing a single pulse of the source of laser energy at an initial target spot in the first of the X rows; moving the pulsed laser to a next target spot; firing a single second spot of laser energy at the next target spot along the first of the X rows; firing a successive number of pulses of laser energy until the end of the first of the X rows is reached; returning to the initial target spot; repeating determined steps a selected number of times; moving the laser in a Y direction to the following second X row; repeating the steps until the last target spot at the end of the array has been fired at; and moving the laser to the initial target spot and repeating the above steps until treatment is completed.

In an aspect, the moving of the laser according to the above steps reduces the amount of treatment time.

In a further aspect, the method my further include a hardware console, the console including a user interface, the programmable controller controlling one or more of: the activating of the pulsed laser source; the moving of the pulsed laser source; the selection of: pulsed or CW operation; the power output of the laser device; selecting the pulse width in the pulsed laser regime; selecting the pulse interval; moving the galvo-mirror control; and controlling the pulse duty cycle.

In yet another aspect, the method may further include the step of controlling the pulse duty cycle by the regulating the time extent of the off time of the laser, and using the off time to fire the laser at one or more subsequent spots.

In yet a further aspect, the laser may be programmed by the controller to operate with set on times and set off times, and wherein the laser is controlled by the controller to be activated during the off times to fire at one or more targeted spots, thereby reducing the overall treatment time for all the spots targeted. The ratio of laser off time to laser on times is adjustable by the controller to set a duty cycle percentage, whereby the percentage value of the duty cycle increases with increasing off times.

In an aspect, a method of treating ophthalmic tissues of the retina by the application of subthreshold laser energy in an area of the retina intended to be treated while reducing heating of the ophthalmic tissues, includes: providing a source of continuous wave (CW) laser energy, wherein the CW output laser is movable in two dimensions over an area of the retina; determining the location and dimensions of an area of the retina tissue to be treated; determining a pattern of movement for the movable CW laser energy source in the area of the retina to be treated; turning on the CW laser energy source; positioning the laser to an initial targeted area; moving the CW laser over the determined pattern until all of the determined pattern has been subjected to laser energy application by the CW laser energy source; returning the laser to the initial targeted area; and, repeating sequence of steps X number of times until treatment is completed. A galvo-mirror apparatus may be utilized to move the laser in the two directions.

In another aspect, the method further includes a programmable controller, and wherein the controller is configured to control the on and off times of the laser; the method further includes the controller moving the laser from the initial targeted area through the determined pattern of movement X number of times, whereby the treatment time is reduced from beginning to completion.

In a further aspect, the method further includes a hardware console, the console including a user interface, the programmable controller controlling one or more of: the activating of the CW laser source; the moving of the CW laser source; the selection of: CW operation; the power output of the laser device; and moving the galvo-mirror control.

In yet another aspect, the determined pattern of movement is in X and Y directions or the determined pattern of movement is in other than in X and Y directions.

In yet a further aspect, the percentage duty cycle is less than 20%; the percentage duty cycle is less than 10%; the percentage duty cycle is less than 5%; and wherein the programmed controller sets the percentage duty cycle to between less than 5% and 40%.

### BRIEF DESCRIPTION OF THE DRAWINGS

FIGS. 1A through 1C illustrate drawings and a table involving treatment of the eyes.

FIGS. 2A and 2B illustrate a prior method of eye treatment.

FIGS. 3A through 3C illustrate one embodiment of the present invention.

FIGS. 4A and 4B illustrate a zoomed in version of the invention of FIGS. 3A through 3C.

FIGS. 5A through 5D illustrate a comparison of the functioning of one prior system compared to that of the present invention.

FIGS. 6A and 6B illustrate the operation of the present invention utilizing a continuous wave (CW) laser energy source.

### DESCRIPTION OF THE PRESENT INVENTION

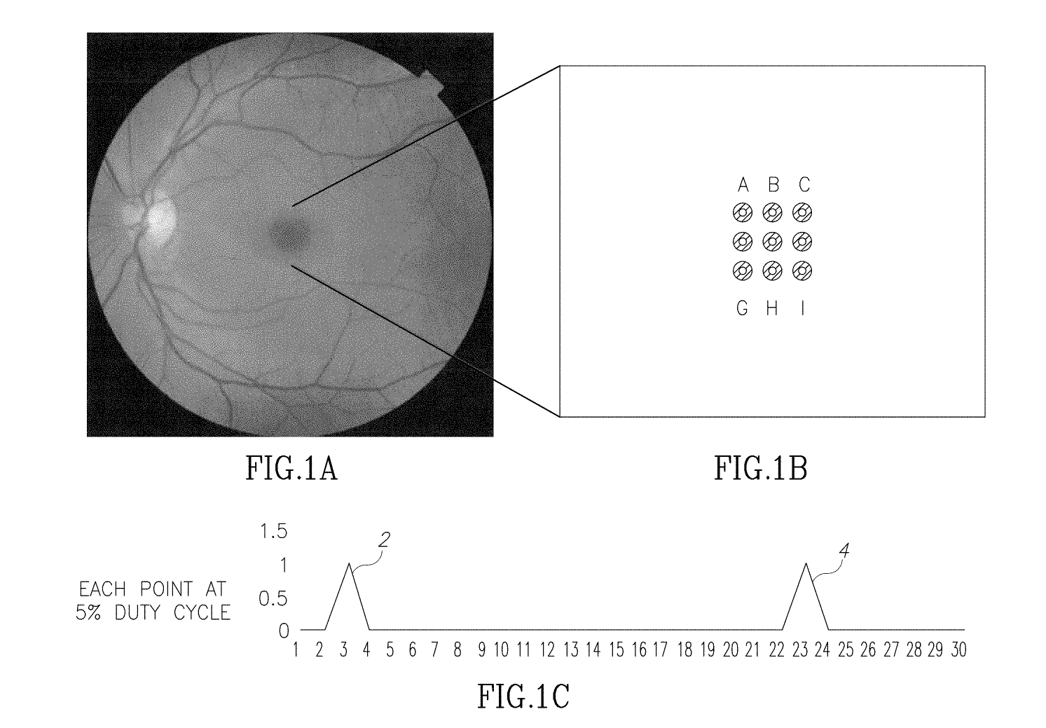

Turning now to FIG. 1A, this figure shows a B&W version photograph of the retina and in the area of the retina which is to receive laser radiation. FIG. 1B shows discrete areas A through I which are representative of spots or areas which are to receive such laser radiation. Although shown as a 3×3 matrix, it is to be understood that any size or shape matrix may be used. In order to avoid complications associated with the destructiveness characteristic of a conventional millisecond continuous wave laser photocoagulation, which causes significant collateral thermal damage, complications such as scotomas, lesion enlargement, subretinal or chorodial neovascularization, fibrosis or progressive visual field loss, it is one aspect of the present invention to provide a subthreshold laser therapy to provide a similar therapeutic effect of lasers while minimizing the damaging effects of lasers.

Subthreshold refers to photocoagulation or photodamage that is insufficient to produce evidence of retinal damage in standard exam such as for example visual examination. It is believed that the therapeutic benefit of subthreshold laser therapy is driven by inducing thermal stress on the Retinal Pigment Epithelium (RPE) cells which are one of the potential absorbers of the laser energy mainly due to their melanin content (other laser energy absorbers may be the choroid as well as the hemoglobin in blood). This thermal stress of the RPE, it is believed, activates the therapeutic cellular cascade. Therefore, the RPE cells need to survive the hyperthermal treatment and accordingly, the goal of the subthreshold therapy is to maintain the temperature rise below the threshold of irreversible thermal damage to the RPE cell.

Heat generation in the tissue is determined by a variety of laser parameters, such as laser spot size, pulse width, duty cycle, power or wavelength. According to the present invention, two strategies to expose the retina to a subthreshold laser treatment are disclosed. According to the first strategy, a pulsed laser is used in conjunction with a laser scanner which is configured to scan a laser beam over a discrete array of treatment spots. According to a second strategy, a continuous laser is used in conjunction with a continuous laser scanner. In both strategies, according to an aspect of this invention, the tissue is exposed to a subthreshold treatment. Accordingly, in another aspect of the invention, a subthreshold laser treatment of the retina is disclosed exposing at least one spot on the retina to the treatment laser for at least one period of time in a microsecond regime. The microsecond exposure regime may be, for example, from 10 microseconds to 1000 microseconds. The tissue spot exposure period may be a single perhaps continuous “on” time. According to another aspect of the invention, a tissue spot on the retina may be exposed to multiple “laser on” times during a treatment session. It is believed that multiple “on” times may be needed, a train of on times, in order to cause a sufficient photoactivation of therapeutic healing response.

According to yet another aspect of the invention, when a train of multiple “on” times is used, cooling intervals between “on” times should be long enough to allow the RPE cells to return to their baseline temperature before the start of the subsequent “on” time. This eliminates cumulative or continuous thermal build-up. The ratio of the “on” time over the cooling period, the “off” time, defines the duty cycle characterizing the treatment.

The two energy strategies discussed above define pulsed and continuous lasers. When a pulsed laser is employed. The “off” time is defined by the time period from the end of one laser pulse until the subsequent laser pulse. In a continuous laser regime, the “off” time may be defined as time it takes for a scanned continuous laser beam (according to whatever scan pattern is used) to reach again a previously scanned spot on the retina. The laser scan pattern and speed are configured to scan a laser beam over a treatment area on the retina at such a speed and in a pattern so that a certain spot on the retina in this treatment area is exposed to a train of on times in the microsecond regime.

According to another aspect of the invention, a subthreshold laser retinal treatment is disclosed having a duty cycle of 20% or less. According to another aspect of the invention, a subthreshold laser retinal treatment is disclosed having a duty cycle of 10% or less. According to another aspect of the invention, a subthreshold laser retinal treatment is disclosed having a duty cycle of 5% or less.

The present invention may be implemented in a number of available ophthalmic devices that are capable of producing pulses in the microsecond regime. One such device is the SMART532, a 532 nm photocoagulator made and sold by Lumenis Ltd of Israel, the assignee of the present application. The SMART532 produces both continuous wave (CW) and pulsed laser energy, called in the device as “SmartPulse” pulses that produce sub-threshold energy. This device has controls that allow the operator to set a number of parameters, including the “SmartPulse” pulse duration, interval and duty cycle. Thus, the present invention is suited for implementation into the Smart532 device. Related to that device is U.S. patent application Ser. No. 15/783,019, entitled “Laser System Having a Dual Pulse-Length Regime”, assigned to Lumenis Ltd. Such application is herein incorporated by reference in its entirety.

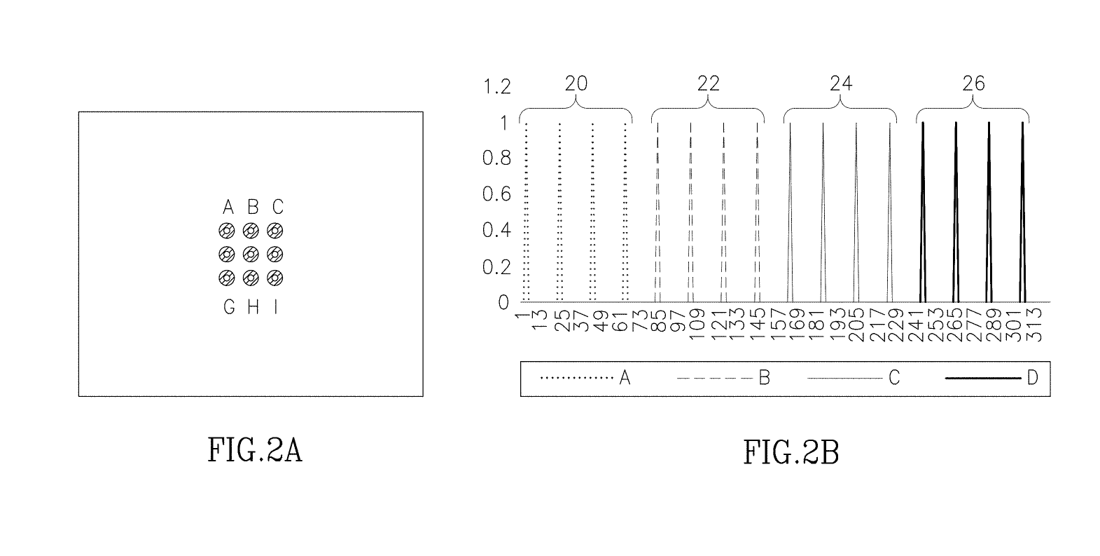

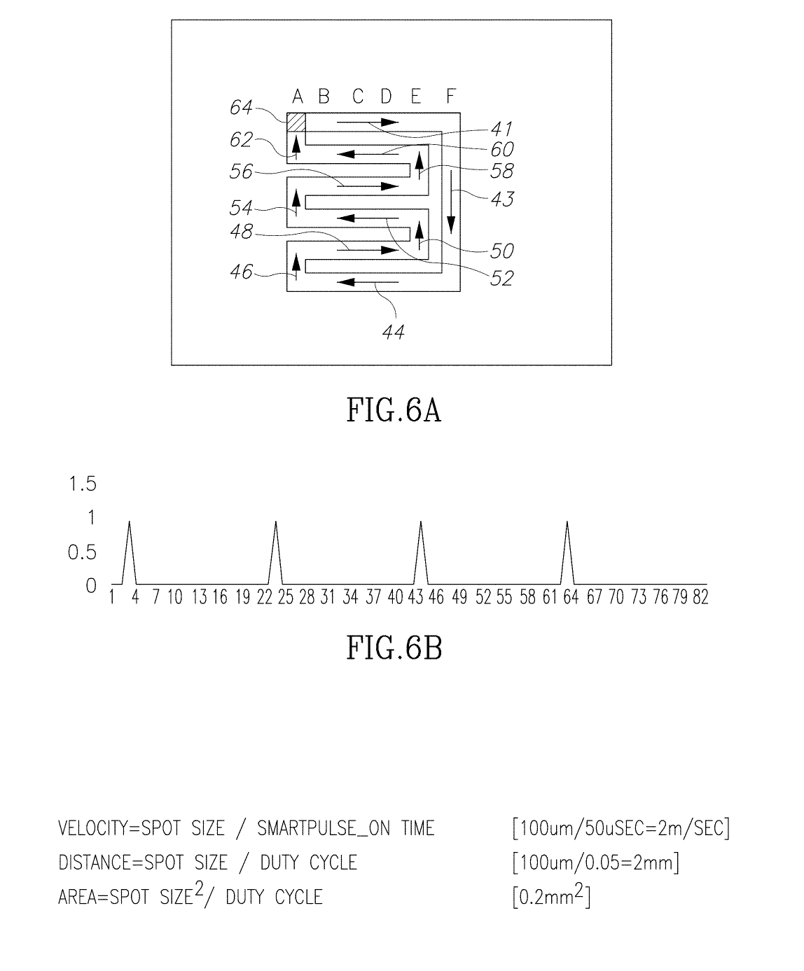

In order to facilitate the movement of the laser, either in the pulsed mode regimes of FIGS. 2 to 5 or the CW regime of FIG. 6, a mechanism such as a known galvo mirror system, may be incorporated into a handpiece to be used with the present system to move the laser in precise movements from targeted spot to targeted spot. One such device is the Array LaserLink, a pattern scanning laser technology made and sold by Lumenis Ltd of Israel, the assignee of the present application.

Further, a programmable controller may be provided to, among other things, control the “on” and “off” times of the laser source, the movement of the galvo-mirror and the movement of the laser from spot position to spot position.

The programmable controller may be mounted in an enclosure or cabinet, the cabinet also containing a visible user interface, suitable processing and memory storage components, and controls to select such functions as: pulsed or CW operation; power output of the laser device; selection of the pulse width in the pulsed laser regime; selection of the pulse interval; galvo-mirror control (as mentioned above); and pulse duty cycle.

In a subthreshold laser treatment discussed, the “off” times are longer than the “on” times. When multiple “on” times are delivered to one spot on the retina, treating multiple spots on the retinal may consume multiple, long “off” times. As a result, the treatment time it takes to treat a patient becomes longer. The fact that during such treatment the patient is preferably in a static position and reduce his/her eye movement further emphasizes the need for a fast treatment.

Therefore, according to another aspect of the present invention which is related to a pulsed energy titration mode, “off” times associated with a first treatment area are utilized to move the scanner to a second treatment area in order to irradiate an “on” time to this second treatment area. Alternatively, the “off” time of a first treatment area may be used to move the scanner to two or more additional treatment areas to further advance the treatment and save more time. An ophthalmic laser system constructed to incorporate the present invention may have a set of one or more duty cycles from which a user can select the required duty cycle for the treatment. For example, such an ophthalmic laser system may include a user interface which may allow the user to select a duty cycle of 5%, 10%, 15% or any other number between 0 to 20%. Therefore, for example, for a given duty cycle of 10%, the “off” time of the laser per a first treatment area is 90%. In order to best utilize the laser during this long off time and in order to accelerate the entire treatment over an area of the retina having multiple treatment spots, the 90% off time may be used to switch the laser to at least one more treatment spot and to irradiate this additional spot with the laser. It may allow to treat up to 9 different spots over the retina during the off time.

Accordingly, a 5% duty cycle may allow to treat up to 20 different spots over the retina during the off time before a second pulse is delivered to an already treated area. This may shorten the treatment time by a factor of about 20 compared to the known irradiation regime known in the prior art in which a laser is “waiting” the entire “off” time on the same spot until it can deliver the second treatment pulse to this same spot. Therefore, according to this aspect of the invention, given a certain duty cycle, the fast scanner and method of scanning and treating is configured to treat additional spots on the retina and to reduce the time of treatment by about a factor of half. Alternatively, if two additional different treatment spots are treated during an “off” time, treatment time will be reduced by a factor of two thirds. Alternatively, if three additional different treatment spots are treated during an “off” time, treatment time will be reduced by a factor of three quarters. Alternatively, if four additional different treatment spots are treated during an “off” time, treatment time will be reduced by a factor of four fifths.

As a general formula, for a given duty cycle DC %, up to additional (100%/DC %−1) treatment areas may be treated during “off” time. Assuming on-time pulse width with a time duration t, at a given duty cycle DC %, in a scan regime as in the prior art where each treatment spot is being treated while waiting the off-time/s on the spot as mentioned above, it would take to treat one treatment spot a time T equal to

T=t×((100%/DC %×NP)−(100%/DC %−1))

where NP is the number of pulses per treatment spot.
For example, if DC % is 25% and NP=1, it would take a time of 1t to treat one treatment spot. If NP=2 it would take 5t to treat a single treatment spot, If NP=3 it would take 9t to treat a single treatment spot and if, for example, NP=4, it would take 13t to treat one treatment spot. Accordingly, if, for example, an array of 4 treatment spots would take 4t, 20t, 36t or 52t respectively to the previous example.

However, according to an aspect of the present invention, the treatment time T per a treatment spot would be:

T=t×NP

Where NP is the number of pulses per treatment spot.

According to this aspect of the invention, the number of spots to treat in this new regime is 100%/DC %. In a duty cycle of 10%, for example, 10 spots would be treated. The spatial distribution of the location of these 10 treatment spots can take shape and sequence within a scanning area of an ocular tissue.

Therefore, at a given DC % of 25%, for example, (100%/CD %−1) additional spots can be treated during off times. Which means that, according to this example, additional 3 spots can be treated so that an array of 4 spots can be treated during one scan. For a single spot it would take 1t to treat the spot. It would take 4t to treat an array of 4 spots with one pulse per spot like in the above example of the prior art regime. However, if NP=2, under this aspect of the present invention, it would take only 8t, if NP=3 it would take only 12t and if NP=4 it would take only 16t to treat an array of 4 treatment spots (compared to the 12t, 24t, 36t and 52t by the old regime). It can be seen that there is a significant time reduction in the time it takes to treat an array of treatment spots for any number of laser pulse per spot which is higher than 1. The more pulses per treatment spot needed, the more time can be saved during a treatment. It should be mentioned that in the discrete energy regime the second, third, fourth, etc., of additional treatment spots being treated during “off” times may be adjacent spots or non-adjacent spots. In a continuous energy titration regime, “off” time is used only to treat adjacent spots which are the next treatment area defined by the scan pattern. In this regime, a treatment spot on the retina is considered to be treated from pulse rise to pulse set as the laser beam moves. Due to the continuous nature of this energy titration mode treatment spots are also continuously gathered along the line of scan.

FIG. 1C shows laser pulses 2 and 4 being applied to the spot marked A on FIG. 1B but spaced apart in 20 units of time, which may be in microseconds. The purpose of such time spacing is to prevent the spot marked A from becoming overheated.

Multiple applications of laser energy to the same spot maybe needed or at least desired to apply an efficacious treatment. In non-ocular applications, it may be possible to apply multiple pulses one after the other in short order since the skin tissue thickness, say at the cheeks, may be of sufficient thickness or depth so as not to be overheated by the multiple applications of laser energy on the same spot or spots. However, in ocular applications, there is the need not to overheat the eye tissue, so the somewhat conventional practice is to hit a spot, wait some period of time for tissue cooling, hit it again repeatedly a selected number of times, while waiting a period of time after each hit (application of a pulse of laser energy). The waiting time between each hit obviously may cause the procedure to become longer than if the multiple hits could be made fairly quickly sequentially.

FIG. 2A shows the same 3×3 matrix of FIG. 1A. FIG. 2B shows a graph of a grouping of multiple laser pulses 20, 22, 24 and 26. While there are shown 4 pulses per grouping, it is to be understood that any suitable number may be chosen depending on the treatment involved. Thus, first four pulses 20 are applied to area spot A, followed by four pulses to area B, followed by four pulses to area C then four pulses to area spot D and so on through spot area I. It can be seen that the pulses in each grouping are separated by approximately 19 units of time to avoid overheating, although the separation time between pulses may be dependent on the treatment involved the patient's condition, etc.

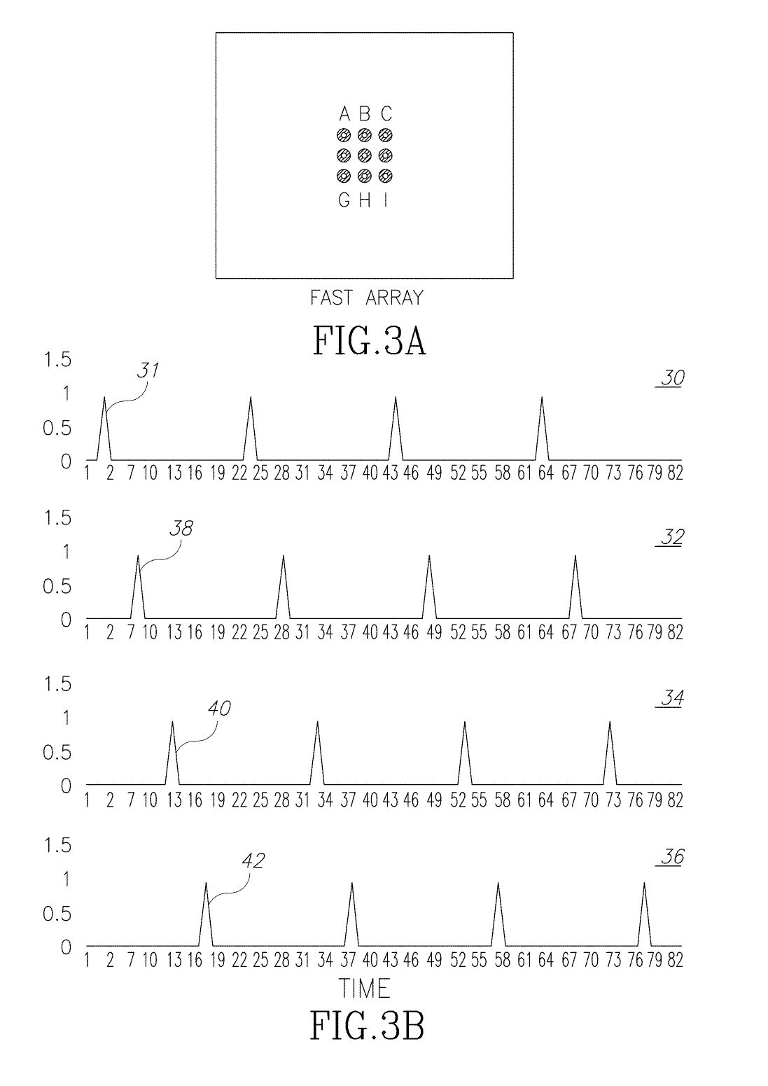

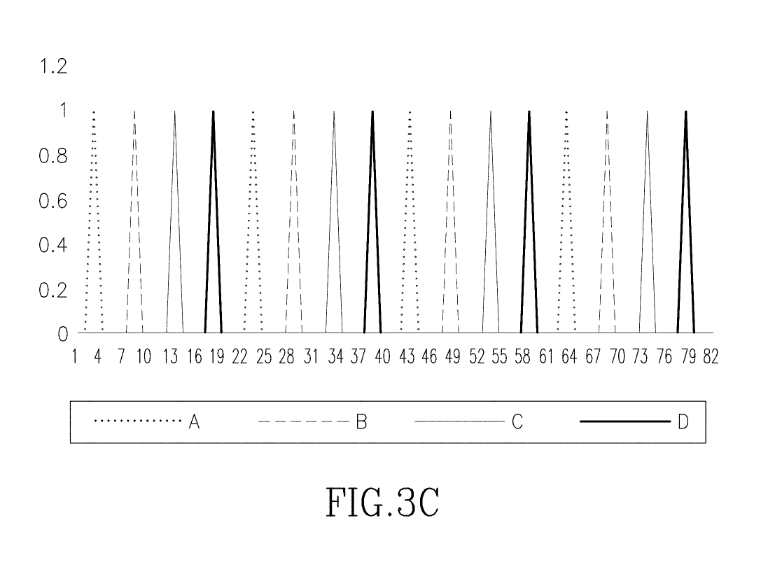

Turning now to FIG. 3, this figure illustrates the structure and timing and placement of laser pulses in accordance with the present invention.

FIG. 3A illustrates the same size matrix as in previous figures. However, FIGS. 3B and 3C illustrate the timing of pulses under what is termed for this invention a “Fast Array”. Here, it is seen in FIG. 3B, reference numerals 30, 32, 34 and 36 that, after a first pulse 31 on spot area A, instead of waiting a period of time as in prior practice and then firing the laser pulse on spot A again, a second pulse 38 is fired on or at spot area B, followed by a third pulse 40 on spot C followed by a fourth pulse 42 on spot area D and so on through spot I in this one illustrative example. However, the present invention in not bound by the foregoing “stepwise” firing of laser pulses, as the pulses can be fired in a random sequence or any sequence to reduce neighbor points from being targeted and thus causing excessive heating of each other. That is, the present invention is not limited to a sequence in which the laser may be made to move only in “X and Y” directions, such as, in FIG. 3A, positions A, then B, then C, then moving down to the next row of positions, but also, by way of example, from position A, then position H, then position C, etc.

The advantage of this firing sequence is that more pulses may be delivered over a given period of time without the danger of overheating the tissue. This is illustrated in FIG. 3C, wherein over a period of about 100 units of time the same number of pulses are delivered as shown in FIG. 1 B except over a much shorter period of time (about 100 units of time vs. about 4×20×3×3=720 units of time). This reduction benefits the patient in that the procedure may be completed more rapidly. In FIG. 3B, point A I identical to FIG. 1C. The reduced time did not affect the treatment of point or position A on the eye or any other point or position on the eye.

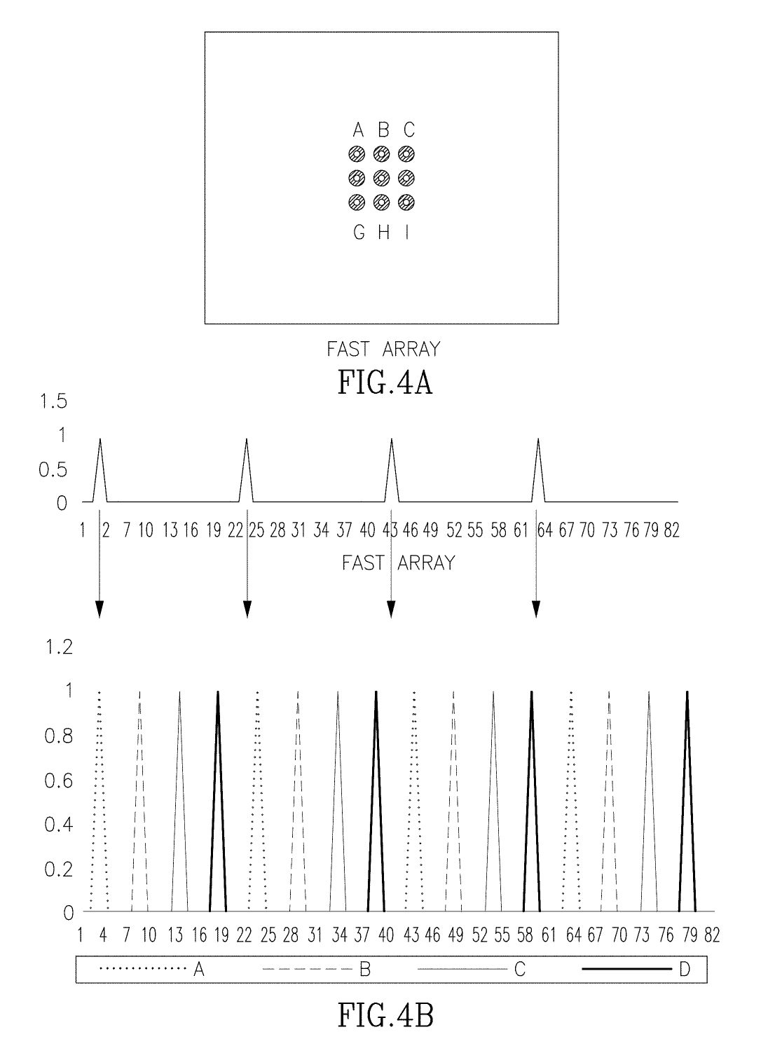

FIG. 4 is similar to and based on FIG. 3 and FIG. 4 B shows, in a zoomed in format, the placement of the pulses for spot A in the same graph as shown in FIG. 3C.

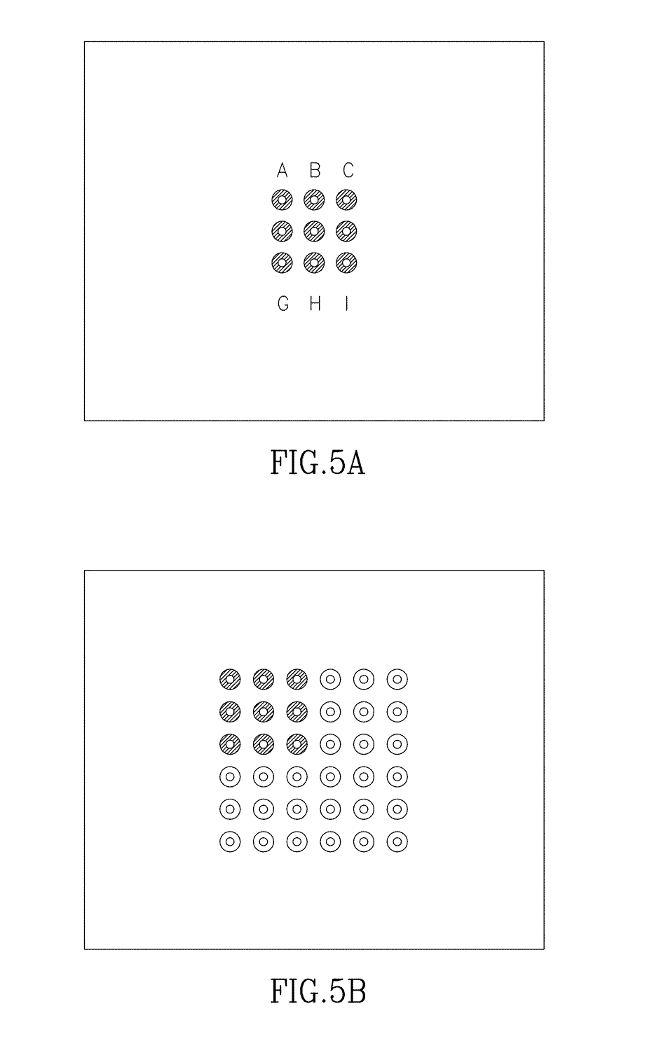

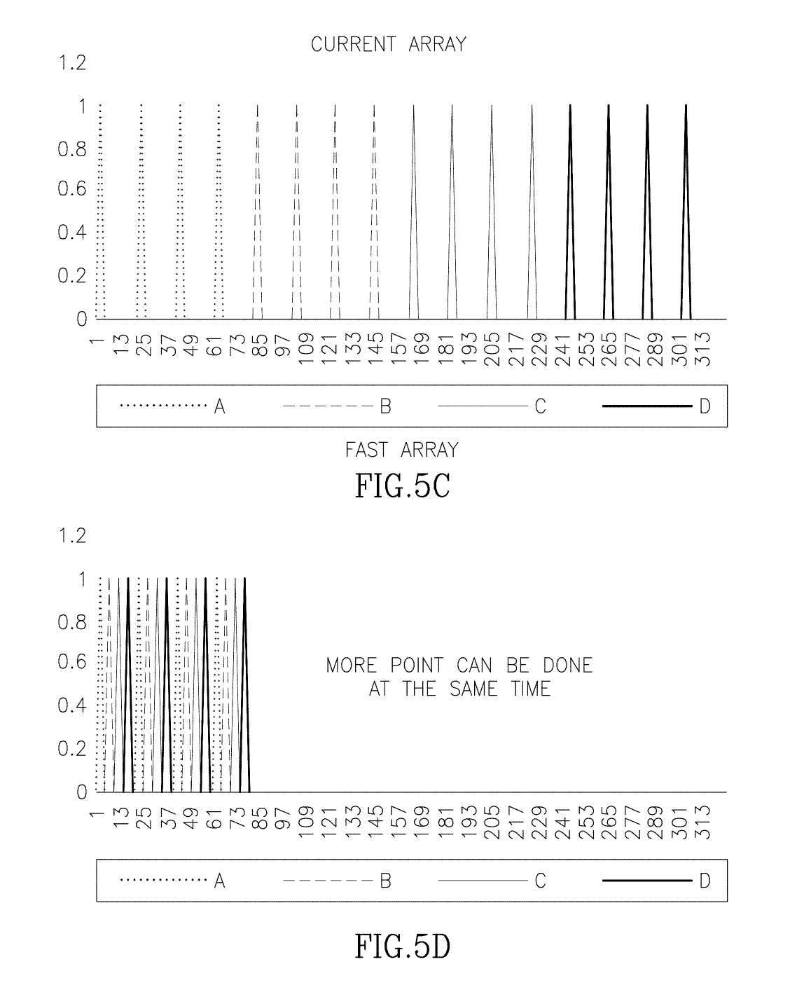

FIG. 5 illustrates a comparison of the current versus the Fast Array technique and the areas of eye tissue that may be targeted and laser radiation applied. Whereas, using the current method in a 3×3 matrix of FIG. 5A over about 700 units of time, each spot area is hit with a pulse as per FIG. 5C in the sequence of FIG. 1, in the same period of time a second (3×3) matrix may be targeted and applied per FIG. 5B since with the Fast Array technique, a 3×3 matrix is completed in about 100 units of time per FIG. 5 D, thus allowing a second 3×3 matrix to be treated in the same amount of time that a 3×3 array is treated under current methods. The upper limit of time saving is 20 times in this example.

Before turning to FIGS. 6A and 6B, it may be mentioned that there are (at least) two different methods of applying laser pulses. One method, which may be termed the “Stop and Shoot” method occurs when the laser is stopped at each spot, applied to that spot, then moved to the next spot under the sequence, stopped and then another pulse applied, and so on.

Another method, which may be termed the “Swap and Shoot” method, is one in which the laser does not stop at each spot on the matrix but rather applies laser pulses while moving from spot to spot, thus further saving time for completing the procedure.

A third method, which may be called “Swap CW”, is one which is perhaps best explained as the application of the present invention to a CW “continuous wave” (versus a pulsed wave) laser system. This is illustrated by FIGS. 6A and 6B.

In FIGS. 6A and 6B, since the laser is always on when activated, it is important that the laser energy will spend exactly one-time unit at each spot area to be treated but then be “moved along” to the next spot. Thus, the laser may be moved in the pattern shown in FIG. 6A in which it is moved from spot A to spots B, D, E and F along the direction of arrow 41 before turning in the directions of arrows 43 through 62 before returning to initial starting point 64 on spot area A. The entire “round trip” from point A back to point A in FIG. 6A is shown to be 20 units of time in FIG. 6B.

Thus, has been described a methodology by which existing devices may be modified or reprogrammed as appropriate to both shorten the amount of time required for a laser eye procedure with the additional benefit of lessening if not eliminating unwanted heating of the eye tissue. The laser can be turned off at specific areas, especially when circling inward.

What is claimed is:

1. A method of treating ophthalmic tissues of the retina by the application of multiple subthreshold laser pulses on each single spot intended to be treated while reducing heating of the ophthalmic tissues, comprising:
providing a source of pulsed laser energy with energy output in the microsecond regime, wherein the pulsed output laser pulse movable in two dimensions;
determining the location and dimensions of an area of the retina tissue to be treated;
determining an array of n target spot positions within the area of the retina to be treated;
(a) targeting a first spot position with one pulse, then targeting the next n spot positions with one pulse until all n spot positions have received one pulse and then restarting the sequence at the first spot position and,
(b) repeating sequence (a) X number of times until treatment is completed.

2. The method of claim 1, wherein a galvo-mirror apparatus moves the laser in the two directions.

3. The method of claim 2, further comprising a programmable controller, and wherein the controller is configured to control the on and off times of the laser; the method further comprising the controller moving the laser from a spot position to a subsequent spot position during the off time of the laser and activating the laser after movement to the subsequent spot;
whereby the treatment time is reduced from beginning to completion.

4. A method of treating ophthalmic tissues of the retina by the application of multiple subthreshold laser pulses on each single spot intended to be treated while reducing heating of the ophthalmic tissues, comprising:
providing a source of pulsed laser energy with energy output in the microsecond regime, wherein the pulsed output laser pulse movable in two dimensions;
determining the location and dimensions of an area of the retina tissue to be treated;
determining an array of X by Y target spot positions within the area of the retina to be treated;
(a) firing a single pulse of the source of laser energy at an initial target spot in the first of the X rows;
(b) moving the pulsed laser to a next target spot;
(c) firing a single second spot of laser energy at the next target spot along the first of the X rows;
(d) firing a successive number of pulses of laser energy until the end of the first of the X rows is reached;
(e) returning to the initial target spot;
(f) repeating steps (a) through (c) a selected number of times;
(g) moving the laser in a Y direction to the following second X row;
(h) repeating steps (a) through (e) until the last target spot at the end of the array has been fired at;
(i) moving the laser to the initial target spot of step (a) and repeating steps (b) through (h) until treatment is completed.

5. The method of claim 4, wherein the moving of the laser according to steps (a) through (i) reduces the amount of treatment time.

6. The method of claim 3, further comprising a hardware console, the console including a user interface, the programmable controller controlling one or more of: the activating of the pulsed laser source; the moving of the pulsed laser source; the selection of: pulsed or CW operation; the power output of the laser device; selecting the pulse width in the pulsed laser regime; selecting the pulse interval; moving the galvo-mirror control; and controlling the pulse duty cycle.

7. The method of claim 6, further comprising the step of controlling the pulse duty cycle by the regulating the time extent of the off time of the laser, and using the off time to fire the laser at one or more subsequent spots.

8. The method of claim 6, wherein the laser is programmed by the controller to operate with set on times and set off times, and wherein the laser is controlled by the controller to be activated during the off times to fire at one or more targeted spots, thereby reducing the overall treatment time for all the spots targeted.

9. The method of claim 8, wherein the ratio of laser off time to laser on times is adjustable by the controller to set a duty cycle percentage, whereby the percentage value of the duty cycle increases with increasing off times.

10. A method of treating ophthalmic tissues of the retina by the application of subthreshold laser energy in an area of the retina intended to be treated while reducing heating of the ophthalmic tissues, comprising:
(a) providing a source of continuous wave (CW) laser energy, wherein the CW output laser is movable in two dimensions over an area of the retina;
(b) determining the location and dimensions of an area of the retina tissue to be treated;
(c) determining a pattern of movement for the movable CW laser energy source in the area of the retina to be treated;
(d) turning on the CW laser energy source;
positioning the laser to an initial targeted area;
(e) moving the CW laser over the determined pattern until all of the determined pattern has been subjected to laser energy application by the CW laser energy source;
(f) returning the laser to the initial targeted area; and,
(g) repeating sequence of steps (d) through X number of times until treatment is completed.

11. The method of claim 10, wherein a galvo-mirror apparatus moves the laser in the two directions.

12. The method of claim 10, further comprising a programmable controller, and wherein the controller is configured to control the on and off times of the laser; the method further comprising the controller moving the laser from the initial targeted area through the determined pattern of movement X number of times; whereby the treatment time is reduced from beginning to completion.

13. The method of claim 12, further comprising a hardware console, the console including a user interface, the programmable controller controlling one or more of: the activating of the CW laser source; the moving of the CW laser source; the selection of: CW operation; the power output of the laser device; and moving the galvo-mirror control.

14. The method of claim 10, wherein the determined pattern of movement is in X and Y directions.

15. The method of claim 10, wherein the determined pattern of movement is in other than in X and Y directions.

16. The method of claim 9, wherein the percentage duty cycle is less than 20%.

17. The method of claim 9, wherein the percentage duty cycle is less than 10%.

18. The method of claim 9, wherein the percentage duty cycle is less than 5%.

19. The method of claim 9, wherein the programmed controller sets the percentage duty cycle to between less than 5% and 40%.
